(19) United States
(12) Patent Application Publication
(10) Pub. No.: US 2025/0295875 A1
(43) Pub. Date: Sep. 25, 2025

(54) UNINTERUPTIBLE CPAP SYSTEM

(71) Applicant: Zopec Medical LLC, Blaine, MN (US)

(72) Inventor: Strong Shihchuang HUANG, Blaine, MN (US)

(73) Assignee: Zopec Medical LLC, Blaine, MN (US)

(21) Appl. No.: 19/089,266

(22) Filed: Mar. 25, 2025

Related U.S. Application Data
(60) Provisional application No. 63/569,583, filed on Mar. 25, 2024.

(51) Int. Cl. A61M 16/00

(52) U.S. Cl. CPC A61M 16/022; A61M 2205/8206; A61M 2205/8262

(57) ABSTRACT

The present disclosure relates to an uninterruptible CPAP device which can detect a required output voltage for a CPAP device and can adjust an output voltage of a power supply to the detected required output voltage. A power supply circuit can selectively bypass the battery pack and enable the power from the external power source to provide power to the CPAP device.

### CROSS-REFERENCE TO RELATED APPLICATION(S)

This application claims the benefit of and priority to U.S. Provisional Application No. 63/569,583, filed on Mar. 25, 2024, the disclosure of which is hereby incorporated by reference in its entirety. To the extent appropriate, a claim of priority is made to the above-disclosed application.

### BACKGROUND

Continuous Positive Airway Pressure (“CPAP”) machines are used to provide continuous positive air pressure to a user's respiratory tract. Often CPAP machines are operated while a user is asleep to assist in keeping the respiratory tract open, thereby improving sleep. CPAP machines are often plugged into a power source such as wall outlets. However, wall outlets can lose power and as a result the CPAP machine may cease functioning. If the operation of the CPAP machine stops unexpectedly, it can cause the user to wake up suddenly, or the user may experience difficulty breathing.

### SUMMARY

In general terms, this disclosure is directed to an uninterruptible Continuous Positive Airway Pressure (“CPAP”) system. In some embodiments, and by non-limiting example, the CPAP system includes an uninterruptible power supply system that provides the uninterruptible power to a CPAP device.

One aspect is an uninterruptible power supply system comprising: a housing; an input port configured to receive power from an external power source; an output port configured to provide power to a CPAP device; a battery pack contained within the housing; a power supply circuit configured to selectively bypass the battery pack and enable the power from the external power source to provide power to the CPAP device, while allowing power to charge the battery pack of the power supply; and a voltage output detection circuit coupled to the output port and configured to detect a required voltage for the CPAP device connected to the output port.

Another aspect is an uninterruptible continuous positive airway pressure (CPAP) system comprising: a CPAP device including a hose and a mask, the CPAP device configured to provide positive pressure through the hose to the mask; and a power supply including: a housing; an input port; an output port configured to provide power to the CPAP device; a battery pack configured to store power from an external power source received through the input port; a power supply circuit configured to supply a required output voltage; bypass the battery pack and enable the power from the external power source to provide power to the CPAP device, while allowing power to be stored in the battery pack of the power supply; a voltage output detection circuit configured to detect the required voltage for the CPAP device connected to the output port; and a controller configured to control charging and bypass of power within the power supply circuit and voltage output of the output port.

A further aspect is an uninterruptible battery pack comprising: a housing; an input port extending through the housing; an output port extending through the housing, the output port configured to provide power to a continuous positive airway pressure (CPAP) device; a battery pack configured to receive power from an external power source through the input port; a power supply circuit configured to bypass the power supply and enable the power from the external power source to provide power to the CPAP device, while allowing power to be charged in the battery pack of the power supply; a voltage output detection circuit configured to detect a required voltage for the CPAP device connected to the output port; and a controller configured to control charging and bypass of power within the power supply circuit and voltage output at the output port.

Yet another aspect is a method of operating an uninterruptible power supply system including: identifying an output cable received within an outport port; determining a required output voltage for a continuous positive airway pressure (CPAP) device based on the identified cable; and supplying the required output voltage from a battery pack to the CPAP device.

### BRIEF DESCRIPTION OF THE DRAWINGS

FIG. 1 is a perspective view of an example uninterruptible continuous positive airway pressure (“CPAP”) system.

FIG. 2 is diagram illustrating multiple CPAP devices being used within another example of the uninterruptible CPAP system.

FIG. 3 is a perspective view of another example of the CPAP device.

FIG. 4 is a block diagram illustrating the operation of the CPAP device.

FIG. 5 illustrates an example cable kit.

FIG. 6 is an example perspective view of the power supply.

FIG. 7 is an example view of the first side of the power supply.

FIG. 8 illustrates an example block diagram of an example uninterruptible CPAP device.

FIG. 9. illustrates an example schematic of an example power supply circuit.

FIG. 10 illustrates an example received voltage identifying circuit.

FIG. 11 illustrates an example voltage output detection circuit.

FIG. 12 illustrates an example method of supplying a determined required voltage output.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

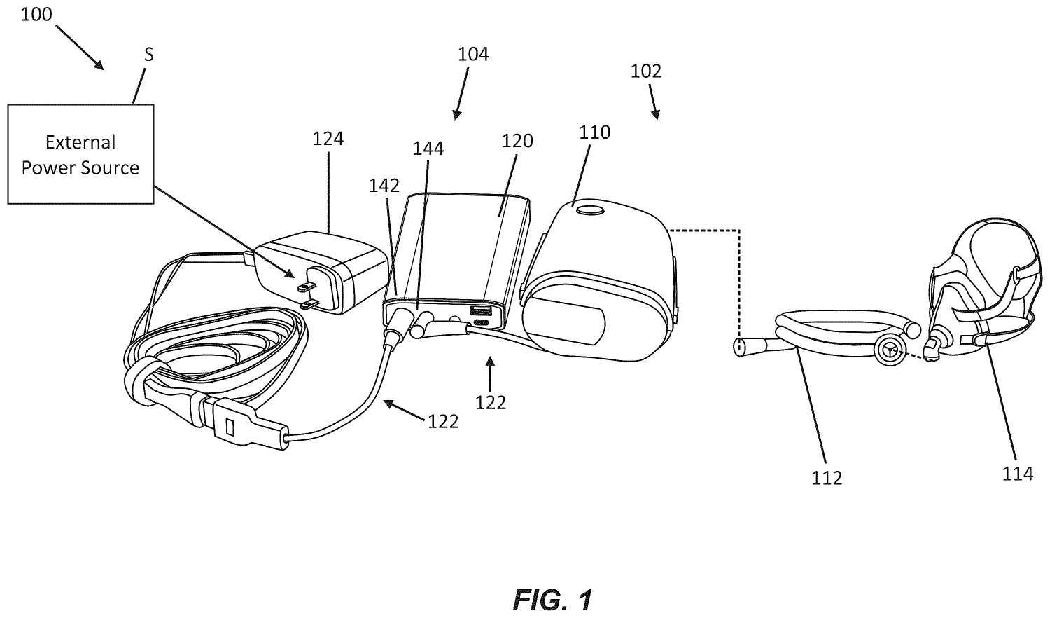

FIG. 1 is a perspective view of an example uninterruptible continuous positive airway pressure (“CPAP”) system 100. The example uninterruptible CPAP system 100 includes a CPAP device 102 and an uninterruptible power supply system 104. The example CPAP device 102 includes a CPAP machine 110, a hose 112, and a mask 114. The example uninterruptible power supply system 104 includes a power supply 120, a cable kit 122, and a power cable 124. The example power supply 120 includes an input port 142 and an output port 144. An external power source S is also shown in FIG. 1.

In the illustrated example, the uninterruptible CPAP system 100 includes a CPAP device 102 and an uninterruptible power supply system 104. The uninterruptible power supply system 104 provides the uninterruptible power to the CPAP device 102 in order to maintain the continuous operation of the CPAP device 102 even in the absence of an external power source S, or in the event that power is lost from the external power source S.

A CPAP device is often worn and used by a user overnight while sleeping. If power is lost while the CPAP device is in use, it can be very disruptive to the user, often causing the user to wake up, and may result in a sensation of sudden difficulty breathing. In order to avoid such disruptions, the uninterruptible CPAP system 100 includes the uninterruptible power supply system 104 that functions to maintain a continuous supply of power to the CPAP device 102 while the device is in use (such as throughout the night), so that the operation of the CPAP device 102 is not interrupted.

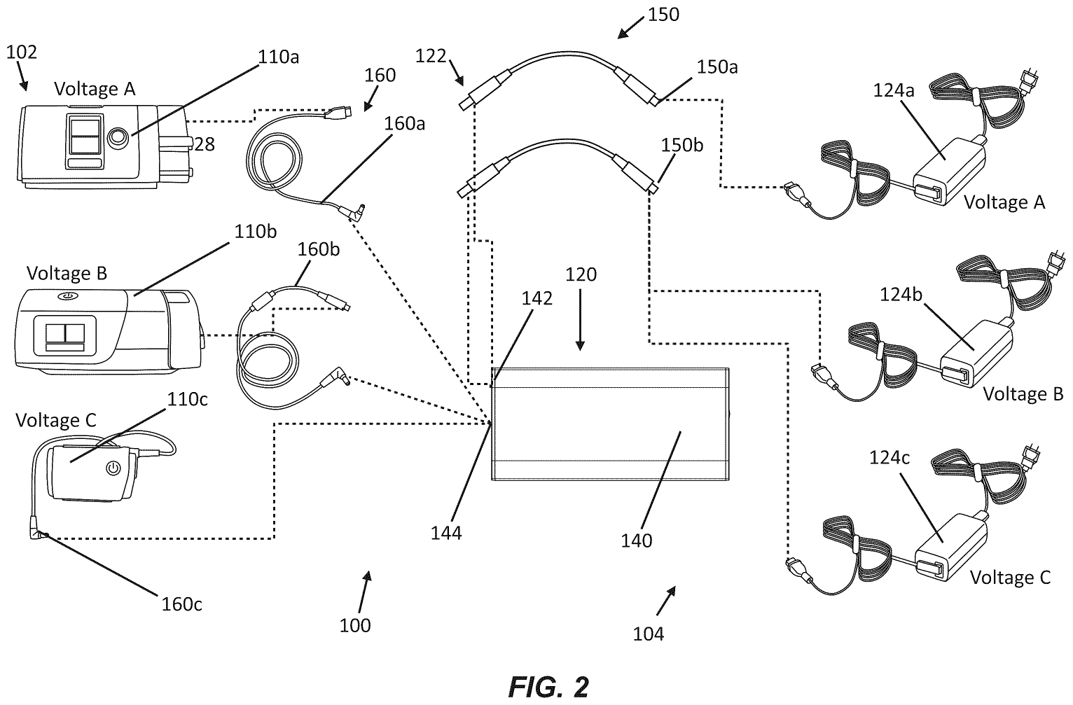

In some embodiments, the CPAP device 102 includes a CPAP machine 110, a hose 112, and a mask 114. The CPAP device 102 operates to provide air to a user at a positive pressure. In some embodiments, the CPAP machine 110 generates pressurized air, and provides the pressurized air through the hose 112 to the mask 114 where it is then delivered to a user. For example, the pressurized air can be provided to the upper respiratory tract of the user, such as through the nose and/or the mouth. Examples of the CPAP device 102 are illustrated and described in further detail herein with reference to FIGS. 2-4.

The uninterruptible power supply system 104 is configured to provide uninterrupted power to the CPAP device 102. The example uninterruptible power supply system 104 includes a power supply 120, a cable kit 122, and a power cable 124. The power supply 120 includes an input port 142 and an output port 144.

The power supply 120 is the portion of the uninterruptible power supply system 104 that receives, stores, and outputs power to the CPAP device 102. In some embodiments the power supply 120 includes a battery pack, which can be charged with power from the external power source S, and can subsequently supply the stored power to the CPAP device 102. In some embodiments, when the power supply 120 is connected to the external power source S, the power supply 120 (such as through the power cable 124, or through the power cable 124 and a cable of the cable kit 122) supplies power to the CPAP device 102, while bypassing the battery pack. In some embodiments, the power supply 120 delivers power from an external power source S and from the battery, such as to collectively provide even greater power output.

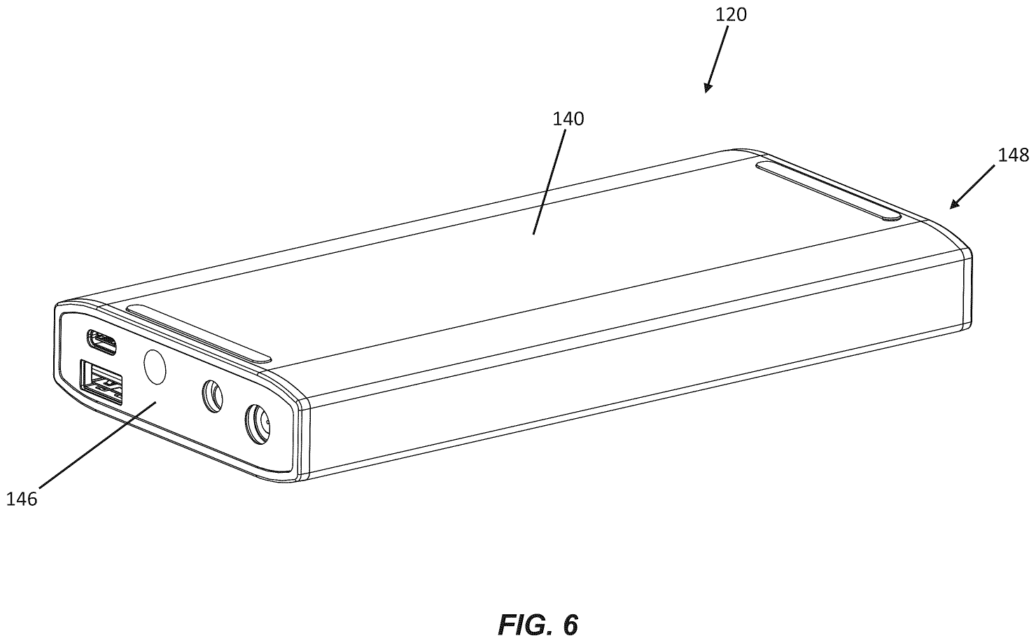

The input port 142 provides a connection for receiving a cable of the cable kit 122 or the power cable 124, to receive power from the external power source S. The output port provides a connection for receiving a cable of the cable kit 122 or a cable of the CPAP machine 110, to supply power from the power supply 120 to the CPAP device 102. The power supply 120 is illustrated and described in further detail with reference to FIGS. 6-11.

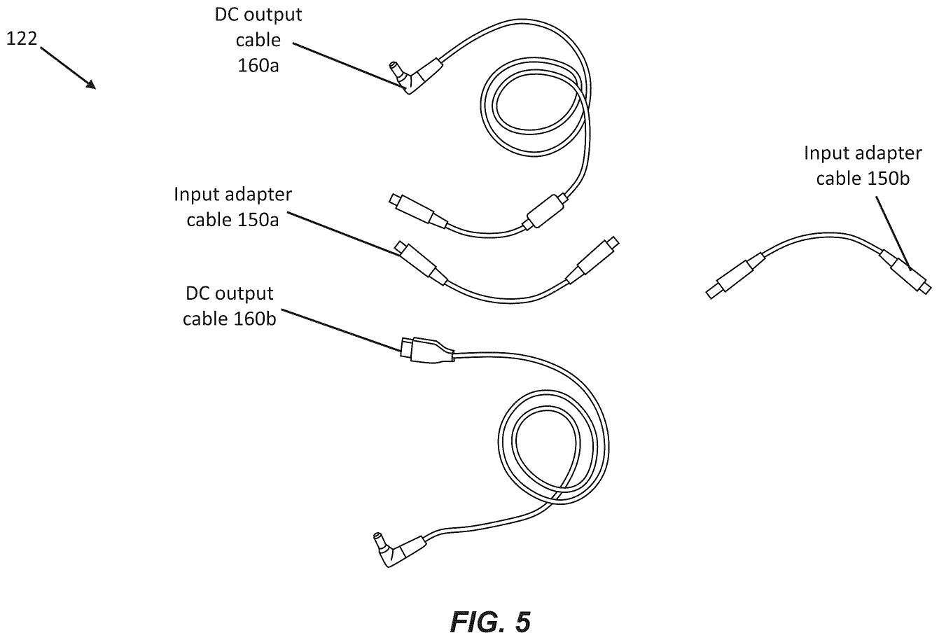

The cable kit 122 includes one or more cables. In some embodiments, an input adapter cable 150 of the cable kit 122 connects the power supply 120 to the power cable 124 at the input port 142. In some embodiments, an output adapter cable 160 of the cable kit 122 connects the CPAP machine 110 at the output port 144. Examples of the cable kit 122 are illustrated and described in more detail with reference to FIGS. 2 and 5.

The power cable 124 receives power from an external power source S for delivery to the power supply 120. In some embodiments, the power cable 124 includes a wall adapter that can be plugged into a wall receptacle supplying mains power. Mains power is typically alternating current (AC), and the wall adapter can include an AC/DC converter that converts the AC power into direct current (DC) power. The DC power is then supplied to the power supply 120, either directly with the power cable 124, or by another cable of the cable kit 122. In other embodiments, the external power source S may be another uninterruptible power supply system 104 to enlarge the available level of stored energy.

The present disclosure refers to example embodiments in which the uninterruptible power supply system 104 provides power to the CPAP device 102. In other embodiments, the uninterruptible power supply system 104 may instead provide power to another electronic device other than the CPAP device 102. An example of another electronic device is a medical device. An example of a medical device is the CPAP device 102. The electronic device can include a device other than a medical device, such as a non-medical device. Accordingly, within the present disclosure, the term CPAP system 100 can, when the context permits, be replaced with the terms electronic system or medical device system to describe other embodiments that are within the scope of the present disclosure. Additionally, the term CPAP device 102, when the context permits, can be replaced with the terms electronic device or medical device to form additional embodiments according to the present disclosure.

FIG. 2 is diagram illustrating multiple CPAP devices 102 being used within another example of the uninterruptible CPAP system 100. In this example, the uninterruptible CPAP system 100 includes a plurality of CPAP machines 110a,b,c and the uninterruptible power supply system 104. In this example, the uninterruptible power supply system 104 includes the cable kit 122 (including a plurality of output adapter cables 160a, 160b, 160c and a plurality of input adapter cables 150a, 150b), the power supply 120, and a plurality of power cables 124a,b,c. A housing 140 of the power supply 120 is also shown.

The uninterruptible CPAP system 100 is configured to allow the uninterruptible power supply system 104 to adapt to a variety of different power requirements of a variety of different CPAP devices 102. For example, in some embodiments the uninterruptible power supply system 104 can identify both a received input voltage from the power cable 124a,b,c and a required output voltage for the CPAP machines 110a,b,c.

FIG. 2 further illustrates the complexity of a possible CPAP system 100, in which the power supply 120 is configured to operate. A CPAP user may often own multiple CPAP machines, such as one or more bedside models, one or more portable-travel models, and the like. In other cases, a user may have both a new and older model of a CPAP machine (i.e., an older model that the user used prior to obtaining the new model). In some situations, each of the CPAP machines 110 may have a different power cable 124 based on the model.

Each CPAP machine 110 and power cable 124 may have different power requirements or characteristics. In some examples, each CPAP machine 110a,b,c may have different voltage requirements to operate. For example, the CPAP machine 110a may require a first voltage A which is different from CPAP machines 110b, 110c which may require different voltages such as a second voltage B and a third voltage C, respectively. As an example, the first voltage A may be 24V, the second voltage B may be 19V, and the third voltage C may be 12V. In other instances, each CPAP machine 110 may have different power supply requirements, such as a required voltage and a required current magnitude. As an example, each CPAP machine 110 may require a minimum current (Amps) or a required power level (Watts). Each unique power supply characteristic may make each CPAP machines 110a,b,c and the power cables 124a,b,c non-interchangeable. As such, by using a power cable 124 which is different from the power characteristics of a CPAP machine 110, the CPAP machine 110 may be damaged, or the power cable 124 may be insufficient to adequately power the CPAP machine 110 during operation. As an example, the power cable 124 may provide a higher voltage to the CPAP machine 110 which could cause damage to the CPAP machine 110. In other instances, the power cable 124 may supply a low voltage which cannot adequately power the CPAP machine 110. In some embodiments, the uninterruptible CPAP system 100 (and the power supply 120) is able to identify the power characteristics of both the power cable 124 and the CPAP machine 110 to safely operate or prevent operation of the CPAP machine.

The power supply 120 is connectable to one of the plurality of CPAP machines 110a,b,c at a time. Each one of the plurality of CPAP machines 110a,b,c has a corresponding output adapter cable 160a,b,c, which connect the CPAP machines 110a,b,c to the output port 144 of the power supply 120. In the example, only a single CPAP machine 110 is connectable to power supply 120 at a time. The input port 142 of the power supply 120 is connected to an input adapter cable 150 at a first end and a second end of the input adapter cable 150 receives one of the plurality of power cables 124a,b,c. It is understood the input adapter cable 150 may also be a plurality of different input adapter cables 150a, 150b, 150c with a matching second end connector to respective power cable 124a,b,c. In the example uninterruptible power supply system 104, the power cable 124a is connectable to the input adapter cable a, while the power cable 124b is connectable to the input adapter cable b. A benefit of the uninterruptible CPAP system 100 is that the power supply 120 is operable to connect and provide power to the plurality of CPAP machines 110 based on the output adapter cable 160 and input adapter cable 150.

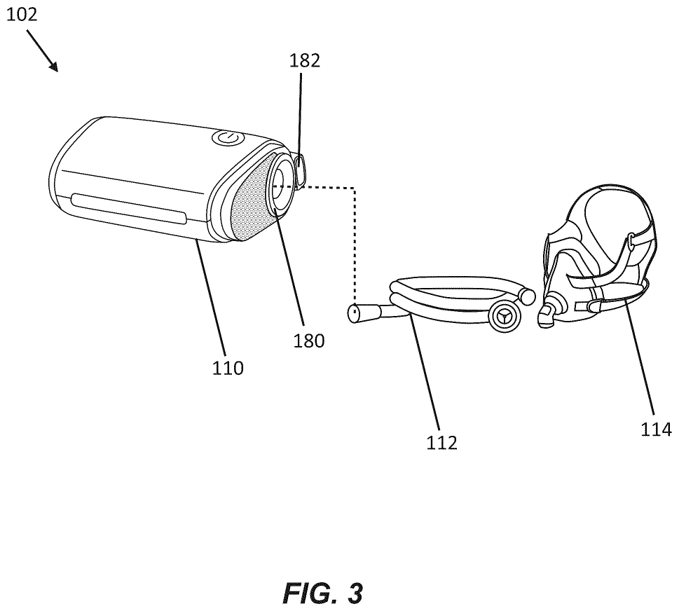

FIG. 3 is a perspective view of another example of the CPAP device 102. The example CPAP device 102 includes the CPAP machine 110, the hose 112, and the mask 114. Additionally, in this example the CPAP machine 110 includes a hose port 180 and a power port 182.

As previously discussed, the CPAP device 102 operates to provide pressurized air to the user. In order to do so, the CPAP device 102 includes a delivery mechanism that delivers the pressurized air to the user. In this example, the delivery mechanism includes a hose 112 and a mask 114.

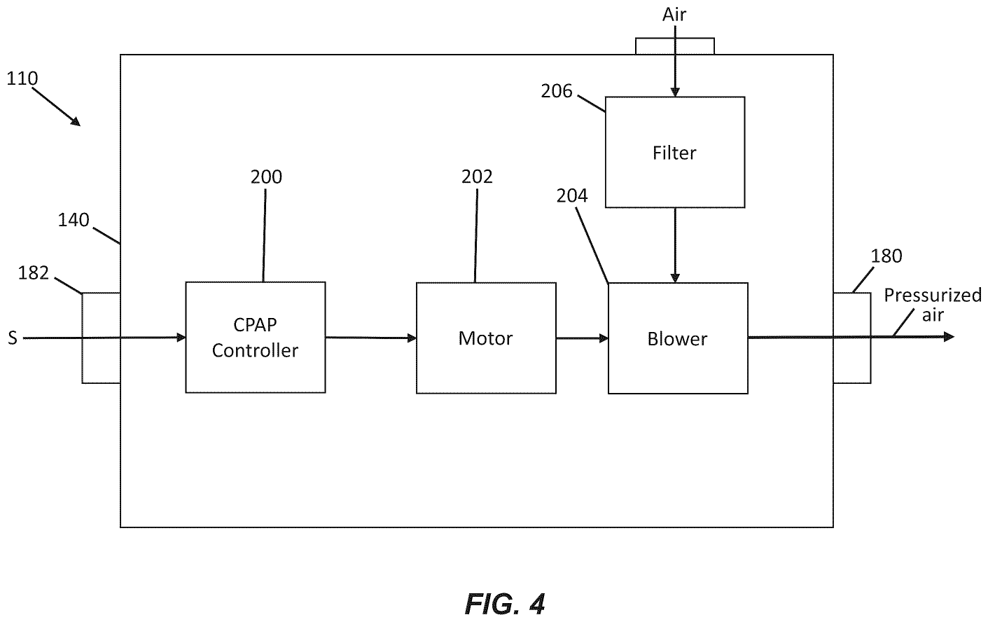

The CPAP machine 110 includes the hose port 180, which is configured to receive one end of the hose 112. During operation, the hose 112 receives the pressurized air generated by the CPAP machine 110 through the hose port 180. An example of the CPAP machine 110 is illustrated and described in further detail with reference to FIG. 4.

In some embodiments, the hose 112 is a hollow elongated conduit with couplers on each end. A first coupler is configured to connect to the hose port 180. A second coupler, at the opposite end, is configured to connect to the mask 114 at a mask port (not separately labeled).

In some embodiments the CPAP machine 110 further includes the power port 182. The power port 182 is an electrical connector for receiving the output adapter cable 160. When connected to the output adapter cable 160 (shown in FIGS. 1-2), the power port 182 is electrically coupled to receive power supplied through output adapter cable 160.

FIG. 4 is a block diagram illustrating the operation of the CPAP device 102. The example CPAP device 102 may include a CPAP controller 200, a motor 202, a blower 204, a filter 206, a hose 112, and a mask 114. The CPAP controller 200 is configured to control the pressure of air supplied by the CPAP device 102 and signal actuation of the motor 202 and the blower 204. In some examples, the CPAP device 102 may have a pressure sensor (not shown) for determining the pressure of air supplied by the blower 204. The motor 202 drives the blower 204 to generate the pressurized air to be supplied through the housing 140 and out of the hose port 180. In some examples, the blower 204 may be a fan or a pump. The housing 140 includes a port 182 which allows entry of air to a filter 206. The blower 204 may generate the pressurized air from air received through the filter 206. The pressurized air may be supplied through the housing 140 and out through the hose port 180. In some examples, the pressurized air may be received at the hose 112 through the hose port 180 (shown in FIG. 3). The hose 112 may connect to the mask 114 at the mask port (not separately shown) where a user receives the pressurized air, such as into the upper respiratory tract. In some embodiments, the CPAP machine 110 further includes a humidifier (not shown), that adds moisture to the air.

FIG. 5 illustrates an example cable kit 122. In this example, the cable kit 122 includes a plurality of input adapter cables 150 and a plurality of output adapter cables 160.a,b. The cable kit 122 may include two pairs of cables such as an input adapter cable 150a pairing with a corresponding output adapter cable 160a and an input adapter cable 150b pairing with a corresponding output adapter cable 160b.

As explained previously, the example cable kit 122 allows the connection of a plurality of CPAP devices 102 to the power supply 120. The example cable kit 122 connects the power supply 120 to the CPAP devices 102 and to the power cable 124 (shown in FIGS. 1 and 2). The example cable kit 122 can include a plurality of input adapter cables 150 and a plurality of output adapter cables 160. In the example cable kit 122, the plurality of input adapter cables 150 includes a first input adapter cable 150a, a second input adapter cable 150b, and the plurality of output adapter cables includes a first output adapter cable 160a and a second output adapter cable 160b. The cable kit 122 is provided to connect a power cable 124 to the power supply 120 by the input adapter cables 150. In some instances, the cable kit allows CPAP devices 102 to be connectable to the uninterruptible power supply system 104 through the output adapter cable 160.

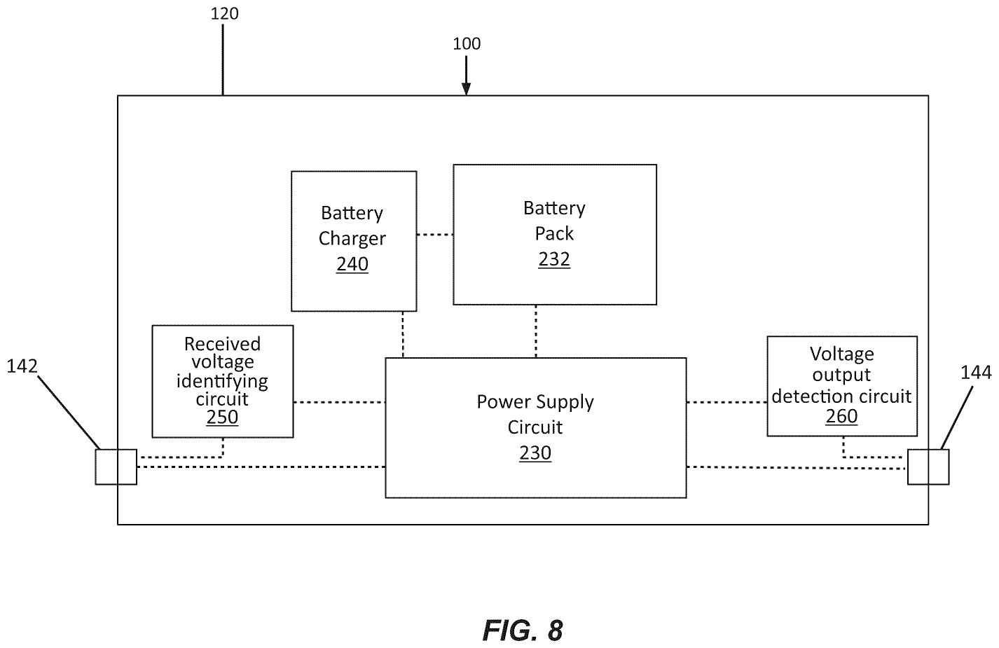

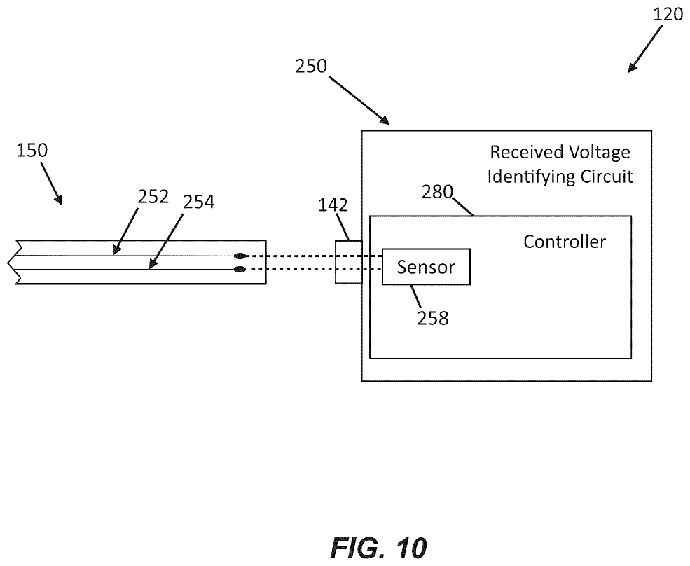

Any number of input adapter cables 150 or output adapter cables 160 may be provided as the cable kit 122, which may allow the cable kit 122 to connect the plurality of CPAP devices 102 to the power supply 120. As discussed previously, the plurality of CPAP devices 102 may include different models of CPAP machines 110 with different power requirements. Here, each of the example input adapter cables 150 is connectable to the power cable 124 (shown in FIG. 1) and to the power supply 120. The input adapter cables 150 may be connected to the input port 142. In some examples, the input adapter cables 150 can provide detectable information of a received input voltage. As will be described in greater detail with reference to FIG. 10, the input adapter cable 150 may include a sensor for detecting the received voltage. The example first and second output adapter cables 160a,b are connectable to the CPAP device 102 and the output port 144 of the power supply 120. Each CPAP device 102 may be associated with a different second output adapter cable 160. The first and second output adapter cables 160a, b provide detectable information of a required output voltage of the CPAP device 102 to be supplied by the power supply 120. The detection of input voltages and output voltages will be described in greater detail with reference to FIGS. 8-11.

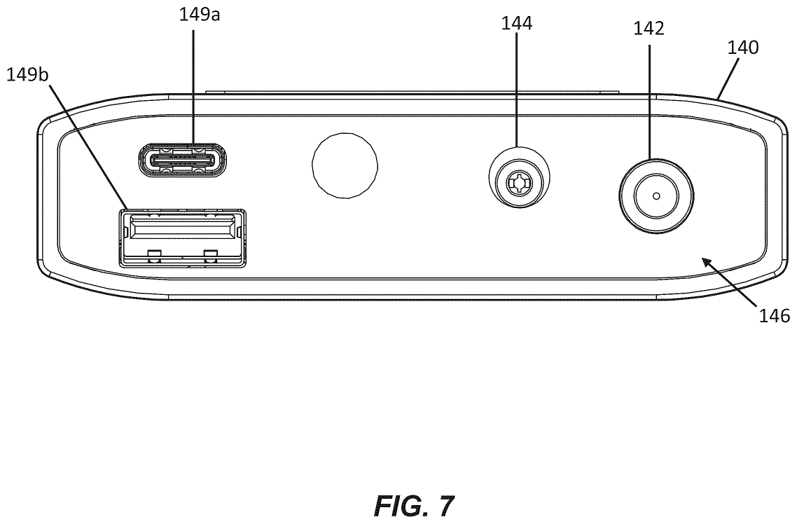

FIGS. 6 and 7 illustrate an example of the housing 140 of the power supply 120. FIG. 6 is an example perspective view of the power supply 120. The power supply 120 may include a housing 140 with a first side 146 and a second side 148. FIG. 7 is an example plan view of the first side 146. The first side 146 includes the input port 142, the output port 144, and a power button. Some embodiments can include additional ports. For example, FIG. 7 illustrates two USB ports 149 as a first USB port 149a and a second USB port 149b. One or more USB ports can be provided in some embodiments. In some instances, anyone of the input port 142, output port 144, power button, the first USB port 149a and the second USB port 149b may positioned on a different side of the power supply, such as the second side 148. The USB ports 149 may be used as outputs to power other devices (i.e. a cell phone). In the example power supply 120, the first USB port 149a is a type C connector and the second USB port 149b is a type A connector. However, other type USB ports may be used. In some examples, the power supply 120 has a weight of no greater than 1.5 lbs, so as to provide a lightweight power supply. In some examples, the power supply 120 has a thickness of no greater than 1 inch, so as to provide a low profile power supply. In some examples, the power supply 120 may select to supply a plurality of output voltages, such as 12V, 19V, or 24V, so as to provide a variable output voltage power supply. Other voltages may be selected and supplied in other embodiments. Other example can have other characteristics greater than, less than, or otherwise different from these characteristics.

FIG. 8 illustrates an example block diagram of an example uninterruptible CPAP system 100. The example power supply 120 includes an input port 142, an output port 144, a power supply circuit 230, a battery pack 232, a battery charger 240, a received voltage identifying circuit 250, and a voltage output detection circuit 260.

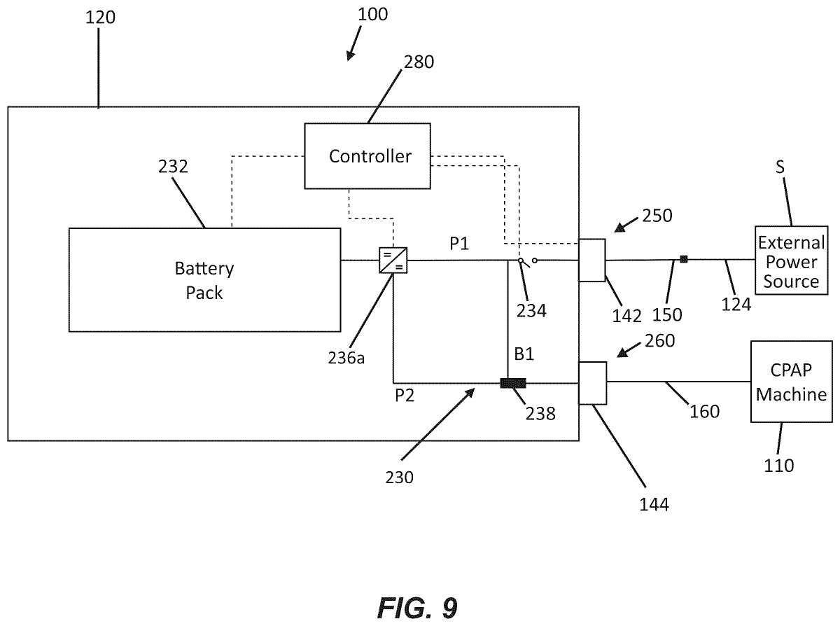

In some embodiments, the power supply circuit 230 operates to charge the battery pack 232 by supplying power received from the input port 142 to a battery charger 240. The battery pack 232 stores powers which can be output to other devices, such as CPAP device 102. The battery charger 240 receives power from the power supply circuit 230 and converts the voltage received to the nominal voltage of the battery pack 232. In the example embodiment, the battery pack 232 may have a nominal voltage of 24V. In some embodiments, the power supply circuit 230 may output power from the battery pack 232 to the output port 144. In some embodiments, the power supply circuit 230 may bypass the battery pack 232 and supply power directly from the input port 142 to the output port 144. Examples of the power supply circuit are illustrated and described in further detail with reference to FIG. 9

The received voltage identifying circuit 250 identifies a received voltage from the power cable 124. An example of the received voltage identifying circuit 250 is illustrated and described in further detail with reference to FIGS. 8 and 10.

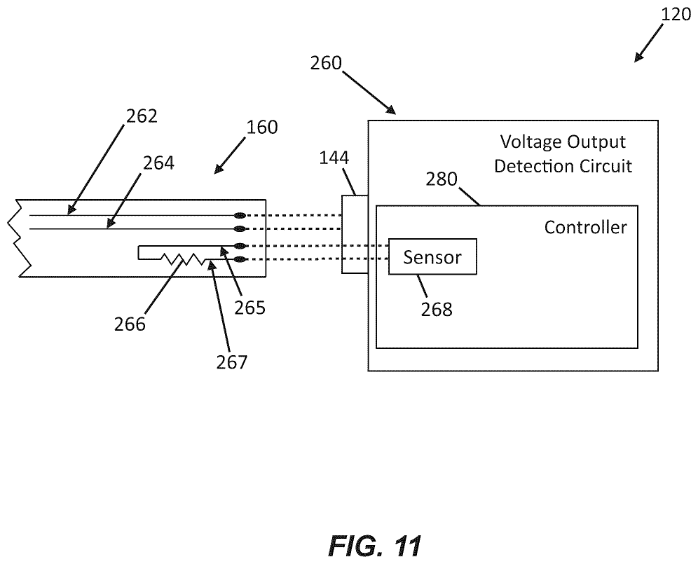

The voltage output detection circuit 260 operates to identify a required voltage to be supplied to a CPAP device 102 through the output port 144. An example of the received voltage identifying circuit 250 is illustrated and described in further detail with reference to FIGS. 8 and 11.

FIG. 9. is a schematic block diagram illustrating an example power supply circuit 230. The example power supply 120 includes a power supply circuit 230, a received voltage identifying circuit 250, a voltage output detection circuit 260, and a controller 280. The power supply circuit 230 includes the input port 142, a battery pack 232, a switch 234, a DC/DC converter 236a, a gate 238, and the output port 144.

The power supply circuit 230 provides a path P1 for received power from the external power source S, and the power cable 124 to charge the battery pack 232, a bypass path B1 for bypassing the battery pack 232, and a path P2 for supplying power from the battery pack 232 to the CPAP device 102. In some examples, the battery pack 232 may include one or more lithium-ion batteries. In other embodiments the battery pack 232 may include other types of rechargeable batteries. In yet another possible embodiment, the battery pack may include replaceable batteries that are not rechargeable.

The received voltage identifying circuit 250 identifies a voltage received from the power cable 124 and communicates with the controller 280 to determine the received voltage. For example, in some embodiments the controller 280 includes a digital voltmeter to measure and determine a voltage at the input port 142.

The voltage output detection circuit 260 determines the required voltage for the CPAP machine 110 and selects a corresponding output voltage to be output by the power supply 120. An example of the voltage output detection circuit 260 is illustrated and described in further detail with reference to FIG. 11.

The controller 280 is configured to control the power supply circuit 230, the received voltage identifying circuit 250, and the voltage output detection circuit 260. The controller 280 may receive information from the voltage output detection circuit 260 to determine the appropriate output voltage. The controller 280 is also configured to detect the available amount of stored power within the battery pack 232. In some examples. The controller 280 may use coulomb counting to determine the amount of stored power available. In other examples, the amount of stored power available within the battery pack 232 may be determined by other methods or means.

The power supply circuit 230 allows the transfer of power through the power supply 120. As an example, the power cable 124 may convert and transfer power from the external power source S through the input adapter cable 150 to the input port 142. For example, the external power source S may supply AC power at 120V, and the power cable may convert the power to DC at 24V or less. The conversion of AC power to DC power within the power cable 124 may be done by an AC/DC converter included within the power cable 124.

When the switch 234 is closed, the power may be supplied through path P1 to charge the battery pack 232. Further power may also be bypassed through path B1, while selectively charging the battery pack 232. In the example embodiment, the controller 280 can control the switch 234 to close to directly supply power from an external power source S to the CPAP machine 110. In some instances, the controller 280 stops charging of the battery pack 232 at the DC/DC converter 236a (i.e., when the battery pack 232 is at full charge), while still bypassing power along path B1. In other examples, the controller 280 is configured to operate a switch on the path P1 to stop charging of the battery pack 232. The switch can be positioned between the path B1 and the battery pack 232 to stop the charging of the battery pack 232.

When the switch 234 is open, the power supply 120 is prevented from receiving power at the input port. As will be described in greater detail with reference to FIG. 10 the controller 280 may operate the switch 234 based on the detection of a voltage received by the received voltage identifying circuit 250. The DC/DC converter 236a may be a two-way converter and may be used to convert the voltage of power entering and exiting the battery pack 232, such as by stepping up or stepping down the voltage.

In the example power supply circuit 230, power supplied along path P1 passes through the DC/DC converter 236a to step-up the received voltage power to 24V. In the example power supply circuit 230, the battery pack 232 can be charged to a nominal voltage of 24V. As an example, the received voltage may be stepped up to 24V by boosting the voltage to charge the battery pack 232 using the DC/DC converter 236a. Power supplied by the battery pack 232 to the CPAP machine 110 passes through a DC/DC converter 236a to step up the supplied power to a required output voltage either by stepping up the voltage or by stepping down the voltage as necessary. An example of the DC/DC converter is a buck-boost converter that operates to step down the received voltage when operating in a buck mode, and operates to step up the voltage when operating in a boost mode. As will be described in greater detail with reference to FIGS. 10 and 11, the controller 280 may control the operation of the DC/DC converter 236a, to select the appropriate mode of operation, and to adjust the step-up or step-down magnitude depending on the received input voltage or the desired output voltage.

The power provided through the DC/DC converter 236a passes through the gate 238 that may act as a switch to selectively provide power to the output port 144. In some instances, the gate 238 may be configured to detect when power supplied through the bypass path B1 has stopped, and then switch to allow power from the battery pack 232 to flow through the DC/DC Converter 236a and the path P2. Additionally, in some examples, the power supply circuit 230 may allow linking of multiple uninterruptible power supply systems 104 together to enlarge an available level of stored power.

FIG. 10 illustrates an example received voltage identifying circuit 250 of the power supply 120, and the input adapter cable 150. In this example, the received voltage identifying circuit 250 includes the controller 280 including a sensor 258. The example input adapter cable 150 includes a positive wire 252 and a ground wire 254. The input port 142 of the power supply 120 is also shown.

The input adapter cable 150 includes one or more wires, such as a positive wire 252 and a ground wire 254. When the input adapter cable 150 is connected to the input port 142, and the opposite end is connected to the power cable 124 (which is plugged into external power source S), the positive wire 252 has a DC signal present on it at a positive DC voltage.

The received voltage identifying circuit 250 may connect with the input adapter cable 150 when inserted the input adapter cable 150 is inserted into the input port 142. The received voltage identifying circuit operates to detect the DC voltage present on the input adapter cable, such as by measuring the voltage of the positive wire 252.

In some embodiments, the received voltage identifying circuit 250 includes the controller 280. In this example, the controller 280 includes a sensor 258. An example of a sensor 258 is a voltage meter, such as a digital voltage meter. The sensor measures the voltage at the positive wire 252, or more specifically the voltage across the positive wire 252 and the ground wire 254. Based on the measured voltage, the controller 280 determines the received voltage.

In some embodiments, the determination of the received voltage includes determining one of a plurality of voltages that are closest to the detected voltage. For example, in one example the controller 280 can be programmed to select from a first voltage (e.g., 24V), a second voltage (e.g., 19V), and a third voltage (12V). The controller 280 selects the first, second, or third voltage based upon which one is closest to the received voltage. In some embodiments the detected voltage must be within a predetermined voltage range of the selected voltage (e.g., within one volt).

As discussed herein, once the received voltage is determined, the controller 280 may control operation of the switch 234 (FIG. 9) to allow charging and/or bypass of power from the external power source S and power cable 124. In some instances, the controller 280 may compare a required voltage output of the voltage output detection circuit 260 to the received voltage of the received voltage identifying circuit 250, as discussed in further detail herein.

FIG. 11 illustrates an example voltage output detection circuit 260 of the power supply 120, and the output adapter cable 160. In this example, the voltage output detection circuit 260 includes the controller 280 including a sensor 268. The example output adapter cable 160 includes a plurality of wires including a positive wire 262, a ground wire 254, a first resistor wire 265, a resistor 266, and a second resistor wire 267. The output port 144 of the power supply 120 is also shown.

The output adapter cable 160 includes one or more wires, such as including the positive wire 262 and the negative wire 264, which function as the primary power delivery wires of the output adapter cable 160. Specifically, the positive wire 262 is configured to receive output power supplied by the power supply 120 and convey the output power for use by the CPAP device 102.

Additionally, the output adapter cable 160 further includes a resistor 266. A purpose of the resistor 266 is to identify the output adapter cable 160, which in turn conveys to the power supply 120 the appropriate output characteristics needed by the associated CPAP device 102. In this example, a resistor having a predetermined resistance (or range of resistances) is provided in the output adapter cable 160. The predetermined resistance (or range of resistances) is an example of a resistive value that can be provided by the output adapter cable 160. The resistor 266 is electrically connected to the output adapter cable 160 connector, by wires such as the first resistor wire 265 and the second resistor wire 267. When the output adapter cable 160 is connected to the output port 144, the first resistor wire 265 and second resistor wire 267 are electrically coupled to the voltage output detection circuit 260, which permits the voltage output detection circuit to identify the output adapter cable 160 using the sensor 268 and the resistor 266. Different output adapter cables 160 have different resistors 266, which have different resistance values.

In one example, the sensor 268 is a current sensor. A known voltage is applied across the resistor 266 at the resistor wires 265 and 267, and the resulting current is detected by the current sensor. Based on the measured current, the controller 280 identifies the output adapter cable 160, such as by use of a lookup table or by otherwise programming criteria into the controller 280. In some embodiments ranges of currents are specified in the lookup table, and in another embodiment the controller 280 identifies a closest current to in the lookup table to the measured current.

In another example, the sensor 268 is a voltage sensor, in which case a similar process is used but with a constant current being supplied to the resistor 266, and a resulting voltage being measured by the sensor 268. The relationship between resistance, voltage, and current is specified by Ohm's law, and as a result variations of the above can be made to arrive at other embodiments consistent with the present disclosure.

Once the output adapter cable 160 is identified, a corresponding required output voltage can be determined by the voltage output detection circuit 260. For example, each of the various resistors used in various output adapter cables 160 can be associated with a given required output voltage. The required output voltage may then be selected by the voltage output detection circuit 260, for delivery by the power supply 120. For example, the controller 280 may adjust the output voltage from the battery pack 232, if needed, to match the required output voltage. In some embodiments, the power supply 120 (and power supply circuit 230) determine whether the input voltage matches the required output voltage. If so, the power supply 120 directly supplies power from the power cable 124 to the CPAP machine 110.

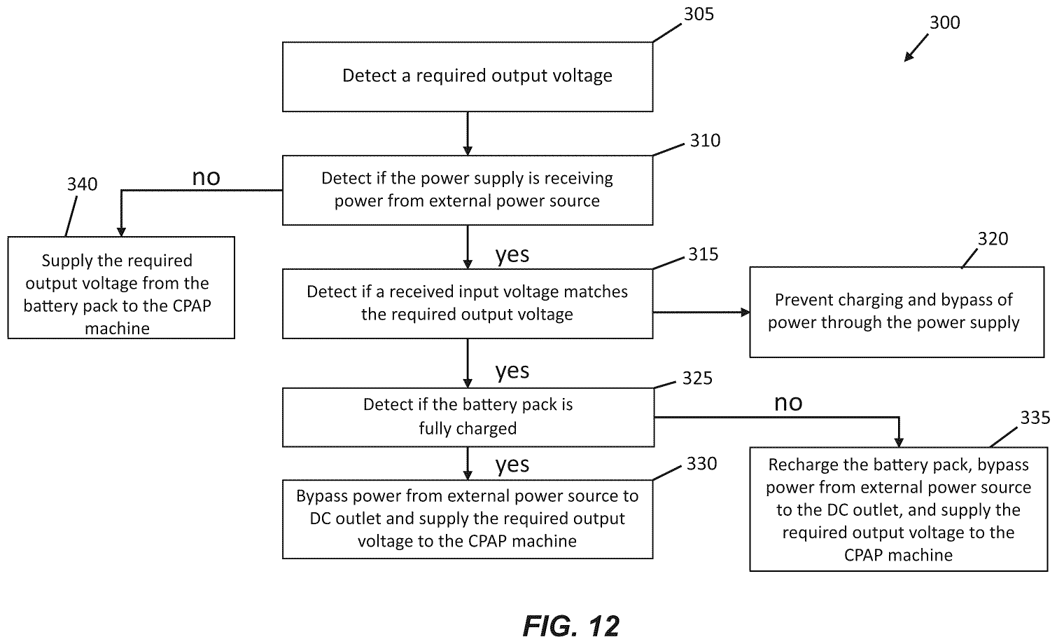

FIG. 12 illustrates an example method 300 of supplying a determined required voltage output. The method 300 includes steps 305, 310, 315, 320, 325, 330, 335, 340, and 345. The step 305 is performed to determine the required output voltage. For instance, the controller 280 may determine the required output voltage through the voltage output detection circuit 260.

The step 310 is performed to detect if the power supply 120 is receiving power from the external power source S. For instance, the gate 238 may detect if power is received from the bypass path b1. If power is not received, step 345 may be performed. Step 345 is performed to supply the output voltage from the battery pack at the required voltage detected in step 305. If power is received, the method 300 proceeds to step 315.

The step 315 is performed to detect the received voltage at the input port 142 and compare to the received voltage to the required output voltage. The received voltage must match the require voltage to prevent providing excess voltage to the CPAP machine 110. If the received voltage does not match the required output voltage, the method 300 proceeds to step 320. Step 320 is performed to open the switch 234 to prevent both charging and bypass of power through the power supply. If the received voltage matches the required output voltage, the method 300 proceeds to step 325.

Step 325 is performed to detect if the battery pack 232 is fully charged. If fully charged, the method 300 proceeds to step 330. Step 330 bypasses power from the external power source S to the output port 144 and varies the voltage of the bypassed power to the required voltage.

If not fully charged, the method 300 proceeds to step 335. Step 335 is performed to allow power to charge the battery pack 232, bypass power from the external power source S to the output port 144 and supply the required output voltage to the CPAP device 102. It is to be understood that steps 320, 330 and 335 may be part a control loop and as such may return to step 305.

The various embodiments described above are provided by way of illustration only and should not be construed to limit the claims attached hereto. Those skilled in the art will readily recognize various modifications and changes that may be made without following the example embodiments and applications illustrated and described herein, and without departing from the full scope of the following claims.

What is claimed is:

1. An uninterruptible power supply system comprising:
a housing;
an input port configured to receive power from an external power source;
an output port configured to provide power to a continuous positive airway pressure (CPAP) device;
a battery pack contained within the housing;
a power supply circuit configured to selectively bypass the battery pack and enable the power from the external power source to provide power to the CPAP device, while allowing power to charge the battery pack of the power supply system; and
a voltage output detection circuit coupled to the output port and configured to detect a required voltage for the CPAP device connected to the output port.

2. The uninterruptible power supply system of claim 1, wherein the voltage output detection circuit includes a sensor within the output port configured to identify the required voltage of the CPAP device.

3. The uninterruptible power supply system of claim 2, further comprising a cable connecting the output port to the CPAP device, the cable includes a resistor providing a resistive value detectable by the sensor, and the sensor determining the required voltage of the CPAP device or identifying a model of the CPAP device based on the resistive value.

4. The uninterruptible power supply system of claim 3, wherein the power supply system supplies the required voltage to the CPAP device based on the resistive value.

5. The uninterruptible power supply system of claim 1, wherein the housing further includes at least one USB type C adapter port, at least one USB type A adapter ports, or both a USB type C adapter port and a USB type A adapter port.

6. The uninterruptible power supply system of claim 1, wherein the power supply system has a weight of no greater than 1.5 lbs, has a thickness of no greater than 1 inch, and supplies power of 12V, 19V, or 24V.

7. The uninterruptible power supply system of claim 1, wherein the power supply circuit is configured to allow linking of multiple of uninterruptible power supply systems to enlarge an available level of stored power.

8. The uninterruptible power supply system of claim 1, wherein the battery pack includes a lithium-ion battery.

9. The uninterruptible power supply system of claim 1, wherein the power supply circuit is configured to detect a level of stored power available within the power supply system.

10. The uninterruptible power supply system of claim 3, wherein the input port is configured to receive an input adapter cable, and wherein when the input adapter cable is inserted into the input port, the input adapter cable is electrically connected to a received voltage identifying circuit that determines a received voltage present on the input adapter cable.

11. The uninterruptible power supply system of claim 10, wherein the received voltage identifying circuit is in communication with a controller configured to identify the received voltage and further configured to control charging and supply of power from the power supply system.

12. The uninterruptible power supply system of claim 11, wherein the controller is further configured to allow the external power source to supply power when the received voltage and the required output voltage match.

13. The uninterruptible power supply system of claim 11, the controller is configured to prevent the external power source to supply power when the voltage received, and the required voltage do not match.

14. The uninterruptible power supply system of claim 1, the power supply circuit further including a switch for allowing power from the external power source to be provided to a bypass path and a path for charging the battery pack.

15. The uninterruptible power supply system of claim 14, the bypass path including a current sensor configured to detect when no power is being received through the input port, and wherein the power supply circuit is in communication with a controller configured to receive signals from the current sensor and upon detection of no power, switch to providing power from the power supply system to the CPAP device.

16. The uninterruptible power supply system of claim 15, the power supply circuit including a DC/DC converter on the path for charging the battery pack.

17. The uninterruptible power supply system of claim 15, the power supply circuit including a switch configured to receive or prevent flow of power from the external power source and a gate connected to the bypass path and a circuit path for supplying power from the battery pack through the DC/DC converter to the CPAP device, the gate configured to detect to switch to providing power from the battery pack to the CPAP device.

18. An uninterruptible continuous positive airway pressure (CPAP) system comprising:
a CPAP device including a hose and a mask, the CPAP device configured to provide positive pressure through the hose to the mask; and
a power supply including:
a housing;
an input port;
an output port configured to provide power to the CPAP device;
a battery pack configured to store power from an external power source received through the input port;
a power supply circuit configured to supply a required output voltage, bypass the battery pack and enable the power from the external power source to provide power to the CPAP device, while allowing power to be stored in the battery pack of the power supply;
a voltage output detection circuit configured to detect the required voltage for the CPAP device connected to the output port; and
a controller configured to control charging and bypass of power within the power supply circuit and voltage output of the output port.

19. An uninterruptible battery pack comprising:
a housing;
an input port extending through the housing;
an output port extending through the housing, the output port configured to provide power to a continuous positive airway pressure (CPAP) device;
a battery pack configured to receive power from an external power source through the input port;
a power supply circuit configured to bypass the battery pack system and enable the power from the external power source to provide power to the CPAP device, while allowing power to be charged in the battery pack of the battery pack system;
a voltage output detection circuit configured to detect a required voltage for the CPAP device connected to the output port; and
a controller configured to control charging and bypass of power within the power supply circuit and voltage output at the output port.

20. A method of operating an uninterruptible power supply system including:
identifying an output cable received within an output port;
determining a required output voltage for a continuous positive airway pressure (CPAP) device based on an identified output cable; and
supplying the required output voltage from a battery pack to the CPAP device.

21. The method of claim 20, further comprising:
receiving an input cable within an input port; and
determining a voltage at the input cable.

22. The method of claim 21, further comprising:
comparing the voltage at the input cable to the required output voltage; and
supplying power from the input port to the output port as a result of the comparison.

23. The method of claim 22, wherein the comparison further comprises determining whether the voltage at the input cable matches the required output voltage.
